(19) United States
(12) Patent Application Publication
(10) Pub. No.: US 2007/0078314 A1
(43) Pub. Date: Apr. 5, 2007

(54) System and method for measuring and predicting insulin dosing rates

(76) Inventors: Richard L. Grounsell, Greenville, SC (US); Robert C. Booth, Columbus, NC (US)

Correspondence Address: ROSENBERG, KLEIN & LEE, 3458 ELLICOTT CENTER DRIVE-SUITE 101, ELLICOTT CITY, MD, 21043, US

(21) Appl. No.: 11/529,224

(22) Filed: Sep. 29, 2006

Related U.S. Application Data
(60) Provisional application No. 60/808,356, filed on Jul. 31, 2006.
(60) Provisional application No. 60/760,156, filed on Jan. 19, 2006.
(60) Provisional application No. 60/721,902, filed on Sep. 30, 2005.

(51) Int. Cl. A61B 5/00

(52) U.S. Cl. 600/319

(57) ABSTRACT

The method and system for managing a patient's blood glucose level predicts an insulin dosing rate to bring a patient's blood glucose level into a preferred target range within a predetermined time interval. The system includes a processor which actuates a blood glucose computer program to measure and predict the patient's blood glucose level. An input mechanism allows for insertion of a preferred target range of the patient's blood glucose level and further permits input of various patient data parameters. The processor calculates the optimum insulin dosing rate for the patient based upon the type of insulin dosing whether it be intravenous dosing and/or subcutaneous dosing. A display mechanism displays the patient dosing parameters and an alarm mechanism alerts a user when the patient's blood glucose level is outside of the preferred patient blood glucose target range.

### BACKGROUND OF THE INVENTION

Maintaining blood glucose levels of a patient within a preferred target range is extremely important in the physical well being of a patient. Blood-glucose levels of a patient may be external to a preferred target range for a patient due to a number of factors including a genetic abnormality, trauma due to injury, and conditions arising from surgical procedures as well as a number of other physical factors.

High blood glucose levels are defined as hyperglycemia which may occur at any time when a patient's blood glucose level is above a preferred target range. Hyperglycemia may be caused by having too much glucose and/or not enough insulin in the body. Symptoms of diabetes are the same as the symptoms for hyperglycemia where diabetes itself may cause hyperglycemia.

Hypoglycemia may occur at any time when a patient's blood glucose is below a preferred target range and is generally caused by not having enough glucose in the body to bring the patient's blood glucose level into the preferred target range.

The subject invention concept is directed to an automated system which allows for predicting in an optimal manner the insulin dosing rate to bring a patient's blood glucose level into a preferred target range over some predetermined time interval.

Management of a patient's blood glucose level is extremely important in diabetic patients where blood glucose levels outside of a preferred target range may cause serious health complications including heart disease, blindness, kidney failure and extremity amputations.

The treatment of diabetes may differ as to the particular type of diabetes from which a patient may be diagnosed. Dosing rates dependent on the type of diabetes will vary and is an important factor in bringing a patient's blood glucose level into a preferred target range.

Type 1 diabetes has been referred to as insulin-dependent diabetes mellitus or juvenile-onset diabetes, which is developed when the body's immune system destroys pancreatic beta cells which make hormone insulin that regulates blood glucose.

Type 2 diabetes has been previously referred to as non-insulin dependent diabetes mellitus or adult-onset diabetes. This type of diabetes is usually initiated as insulin resistance where the cells do not properly use the insulin provided by the body.

### FIELD OF THE INVENTION

This invention is directed to a system and method for measuring and predicting optimal insulin dosing rates in order to bring a patient's blood glucose level into a preferred target range.

The subject invention is directed to a system having a computer-directed formula system for evaluation of the current as well as cumulative patient blood glucose values. Based upon the aggregate of the measurements computed by the computer system. A calculation is provided and a recommended insulin dosing rate is predicted to drive the blood glucose level of the patient into the predetermined target range.

The subject invention is further directed to a portable system whereby the attending physician and/or caregiver is given an alarm or otherwise alerted to the fact that the patient's blood-glucose level is external to a preferred target range.

The subject invention further relates to a system and method whereby information derived from the calculated blood-glucose dosing rate may be transmitted automatically to an external station which may be through a wireless transmission or a hard linkage to some remote station printer, computer server, or other information receiving system.

The invention relates to a computer-directed formula system for evaluation of optimum blood glucose dosing rates to a patient which includes both intravenous and/or subcutaneous dosing conditions for the patient.

Still further, the subject invention directs itself to a method and system for prediction and management of the blood dosing rate of a patient wherein calculations may be performed as to whether it is a pre-prandial or post-prandial state.

More in particular, the subject invention system and method is directed to which re-evaluates the patient's optimum dosing rate dependent upon prior blood glucose readings and predicts a dosing rate to bring the patient's blood-glucose level within the preferred target range within a predetermined time interval.

Additionally, the subject invention relates to a method and system where a patient's diabetes condition, whether a Type 1 or Type 2 diabetic is taken into account in the prediction of the dosing rate to be administered.

Further, the subject invention directs itself to a method and system where the dosing rate to be administered is calculated to include pre-prandial or post-prandial states.

### PRIOR ART

Various systems and methods for measuring and predicting insulin dosing rates have been used in the prior art. In some prior art predictions, a simple equation of the form of blood glucose level of the patient minus a constant which stayed fixed were multiplied by some type of multiplier which was generally protocol dependent based upon the input of the attending physician or the caregiver. Such prior art methods produced predictions of future time interval blood glucose levels which were far out of range of a patient's standard blood glucose reading.

In some prior art systems and methods, the attending physician or caregiver provides for a dosing rate which is based upon an initial time interval and does not take into account changes in the patient's physical parameters during that time interval leading to an over shoot or under shoot of the blood glucose levels of the patient at the end of the time interval.

In other systems relating to intravenous insulin protocols, there is the disadvantage that the blood glucose levels are required on a variable schedule and are difficult to reproduce without a timing and alarm mechanism.

With respect to other prior art protocols for intravenous insulin dosing, the patient may not take intermittent meals of carbohydrates since this titrates up the insulin dosing rate which then carries on beyond the availability of the substrate.

In other prior art systems, methods and protocols used for predicting blood glucose levels of patients, there is no iterative procedure taken for differing time intervals which leads to a non-optimal dosing rate for the blood glucose dosing rate for the patient.

In other prior art systems and methods, there is no methods for measurements, predictions and protocols for insulin dosing rates, there is no provision made for providing an alarm to the attending physician and/or the caregiver to alert them that a patient's blood glucose level is out of range of the preferred blood-glucose target range.

In some other prior art systems and methods for measuring and predicting blood glucose levels in a patient, there is no provision for the portability of the overall system to allow the attending physician and/or caregiver the ability to permit movability from one patient to another.

In other prior art systems for the measuring and predicting of blood glucose levels in patients, there is no automatic system which transfers the patient's dosing rate data to an external device at a remote station.

In some other methods and systems for measuring and predicting the glucose levels in a patient, there is no ability to transfer between intravenous dosing and subcutaneous dosing at the discretion of the attending physician and/or caregiver.

### SUMMARY OF THE INVENTION

The subject invention is directed to a system for measuring and predicting optimum insulin dosing rate to bring a patient's blood glucose level into a preferred target range in order to more effectively manage a patient's blood glucose levels.

The measuring and predicting system includes a computer-directed formulation system which evaluates the current and well as cumulative patient blood glucose values and then based upon the aggregate of the measurements, calculates and recommends the insulin dosing rate to drive the patient's blood glucose level into the preferred blood glucose target range. The computer-directed formulation system may be applied to various devices, including, for example, IV infusion pumps, insulin pumps, glucose meters and glucose sensors.

The subject system and method includes an iterative process where the patient's blood glucose level is measured at predetermined time intervals and calculates the recommended dosing rate based upon whether the patient is being treated intravenously or subcutaneously.

The subject invention is further directed to a blood-glucose monitoring system where the attending physician and/or care provider is provided with alarms both visual and/or audio when the blood glucose level of the patient is external to the preferred target range.

An object of the subject invention is to provide a system for measuring and predicting an insulin dosing rate dependent upon whether or not an intravenous dosing, a subcutaneous dosing or an intravenous and subcutaneous dosing is applied to the patient.

A further object of the subject invention is to provide a system and method whereby the dosing of insulin is optimized based upon the blood glucose level of the patient and a preferred target range for the blood glucose level.

A still further object of the invention is to provide a measurement and prediction system which is portable in nature and can be coupled to a variety of external computer system through either a direct connection or through a wireless transmission to a remote station.

The subject invention includes a method of measuring and predicting a subcutaneous insulin dosing rate to bring a patient's glucose level into a preferred target range where a processor is established to actuate a subcutaneous computer program for measurement and prediction of the patient's blood glucose level.

The subject invention method includes the input of predetermined patient data into the processor which includes the patient target range as well as the patient's weight and other physical parameters.

The subject invention method is directed to the measurement and prediction of a subcutaneous insulin dosing rate which calculates the optimum insulin dosing rate dependent upon whether the calculation is being made for pre-prandial or post-prandial conditions.

The invention method further includes an optimal measuring and predicting method for intravenous insulin dosing rate in accordance with an intravenous insulin dosing rate formula which includes establishing a processor to actuate an intravenous computer program for measurement and prediction of the patient's glucose level.

The subject invention method for measuring and predicting an intravenous insulin dosing rate includes calculation of the optimum dosing rate of the insulin in accordance with the patient's blood glucose level, a sensitivity factor and constant which is dependent upon whether capillary measurement, arterial measurement, venous measurement or interstitial measurement is being taken.

The subject invention method is still further directed to a method for optimally measuring and predicting the intravenous insulin dosing rate which includes calculation of the optimum dosing rate at predetermined time intervals.

### BRIEF DESCRIPTION OF THE DRAWINGS

FIG. 1 is a schematic block diagram of an exemplary system configuration for carrying out aspects of the present invention;

FIG. 2A is a computer flow diagram of fundamental method steps of an exemplary embodiment of the present invention;

FIG. 2B is a computer continuation information flow diagram in FIG. 2A;

FIG. 3 is a computer flow diagram of fundamental method steps of the express intravenous treatment methodology;

FIG. 4 is a computer flow diagram of fundamental method steps of the subcutaneous methodology;

FIG. 5 is a computer flow diagram of fundamental methods steps of the algorithm from FIG. 2B;

FIG. 6A is a computer flow diagram of fundamental method steps of the IV treatment information page;

FIG. 6B is a computer continuation information of the flow diagram from FIG. 6A;

FIG. 7A is a computer flow diagram of the fundamental method steps of the subcutaneous treatment page; and,

FIG. 7B is a computer continuation of information flow diagram from FIG. 7A.

### DESCRIPTION OF PREFERRED EMBODIMENTS

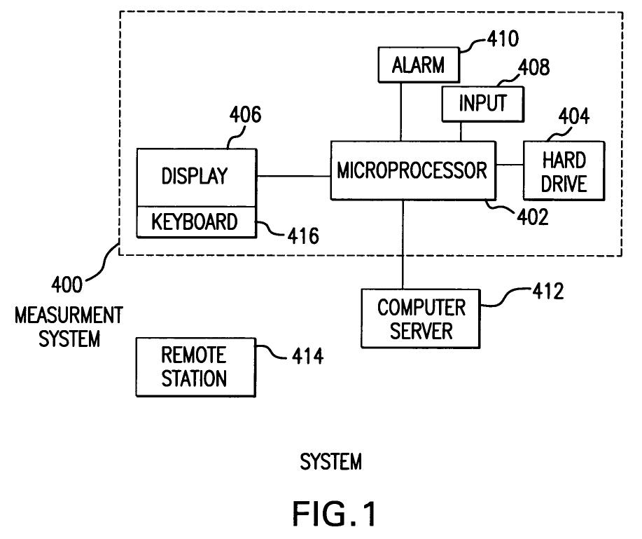

Referring now to FIG. 1 there is shown a diagram for a system 400 for measuring and predicting an insulin dosing rate to bring a patient's blood glucose level into a preferred target range. The measurement and prediction system 400 may be a stand alone system or may be portable to be carried by the attending physician or caregiver from one patient to another, as will be described in following paragraphs.

Measurement and prediction system 400 includes microprocessor 402 which is used for actuating a computer program for measurement and prediction of the patient's blood glucose level and further for storing and maintaining the patient's blood glucose level taken at predetermined time intervals. The microprocessor 402 of the prediction and measurement system 400 may be incorporated with a hard drive 404 and includes a display 406, which may be in the form of an LCD monitor or some other well known type of display system. The display 406 may incorporate keyboard 416 for input of data from data block 408. Alternatively, display 406 may be a touch screen type of input device which is well known in the art. The input mechanism 408 inputs a preferred target range for a particular patient's blood glucose level.

Measurement and prediction system 400 further includes alarm mechanism 410 which alerts a user when the patient's glucose level is external to the preferred patient glucose target range. The alarm may be an audio alarm in the form of a buzzer or some like audio sounding mechanism or may be visual in nature to provide a warning message or other type indicia indication on display 406.

It is to be understood that microprocessor 402, input block 408, alarm 410, hard drive 404 and display 406 with keyboard 416 may all be incorporated into a single handheld unit comprising system 400.

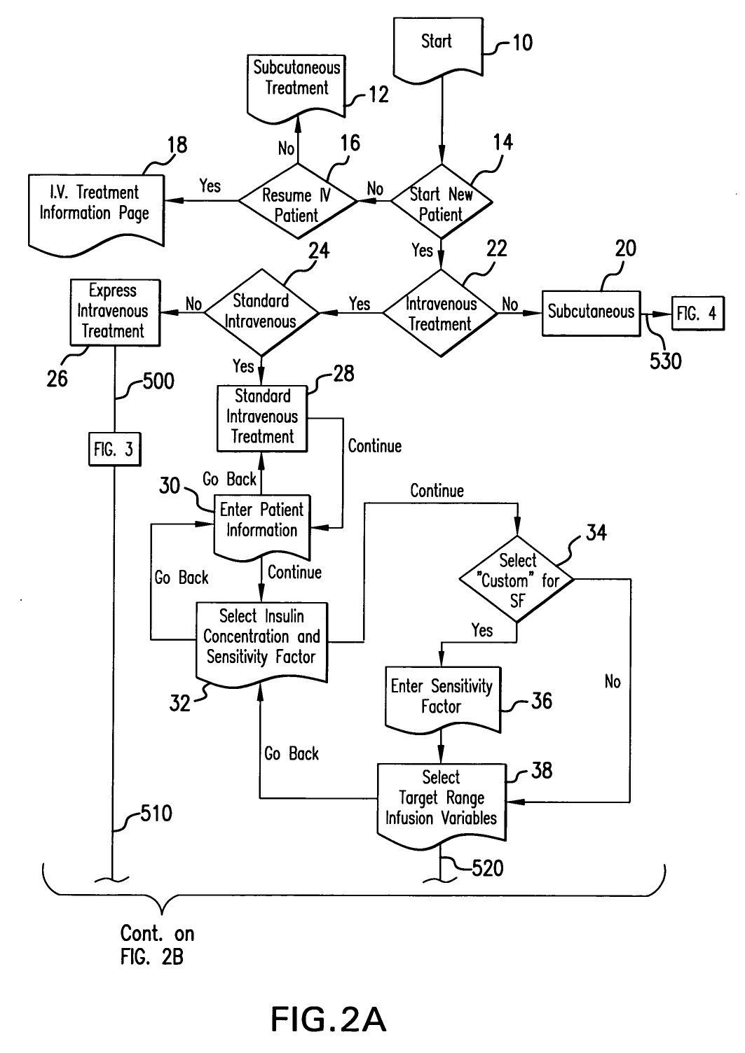

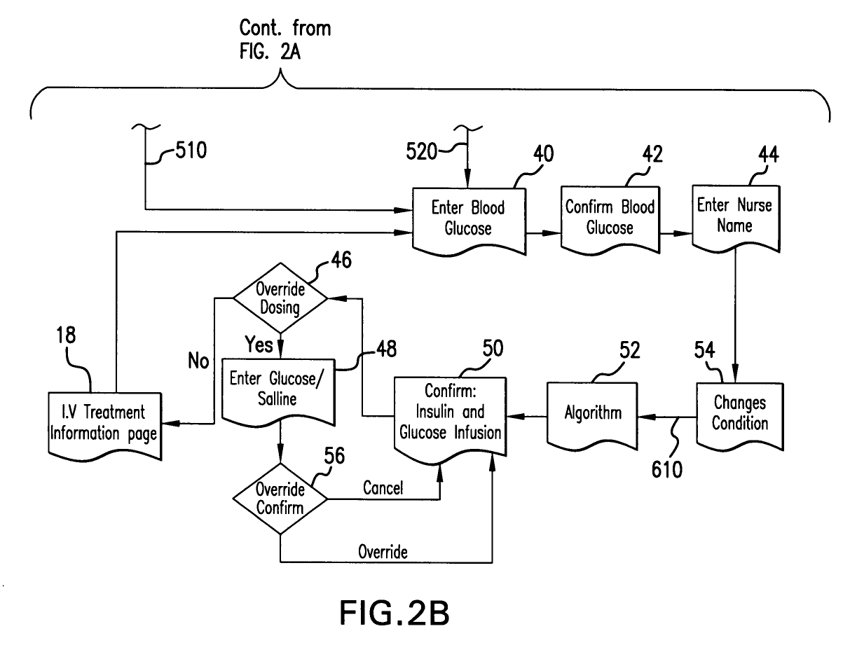

Referring to FIGS. 2A and 2B, there is shown the overall block diagram associated with the prediction and measurement system 400 as provided in FIG. 1. Initially, block 10 refers to a start procedure block where microprocessor 402 and associated display 406 are actuated. Initially, the information flow passes to decision block 14 where there is a decision made as to whether this is the start of a new patient or whether there is a resumption of the IV treatment for the patient which is determined in decision block 16.

Referring again to FIGS. 2A and 2B showing the overall block diagram for computer actuation programs in processor 402, the initialization begins in block 10 where the logic inquires as to whether this is the start of a new patient in decision block 14. If the start of the new patient is to be actuated, logic flows to decision block 22 deciding whether this is an intravenous treatment or subcutaneous treatment. Input may be made on keyboard 46 or directly on display 406 if it is a touch screen actuation.

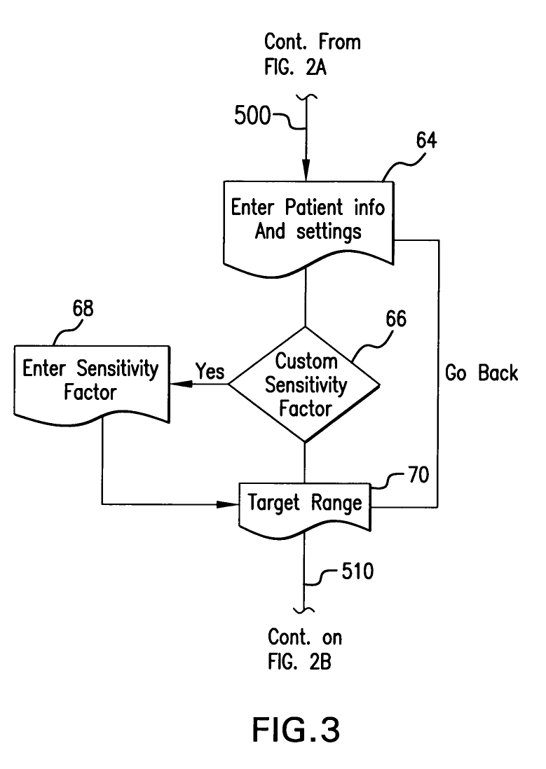

If intravenous treatment is input, logic flow extends to the decision block 24 where it is determined whether a standard intravenous dosing is to be provided or whether an express intravenous treatment is to be entered as shown in block 26. If express intravenous treatment is either chosen by the user or is preset within prediction and measurement system 400, the logic flow passes to FIG. 3 through information line 500 where patient information and settings are entered into microprocessor 402 at logic block 64 (shown in FIG. 3). In the express intravenous treatment provided in block 26 much fewer options are needed to be entered by the attending physician or caregiver than the standard options as will be seen in following paragraphs. In block 64 the patient general ID information is provided including patient ID, age, weight and height. Once entered in logic block 64, the logic flows to decision block 66 where a determination is made as to whether a custom sensitivity factor is to be provided. If a custom sensitivity factor is to be used in the dosing rate, then the logic flows to logic block 68 where the sensitivity factor is entered by the attending physician or caregiver. The initial sensitivity factor is an empirically defined sensitivity factor which may be entered as 0.01, 0.02, 0.03, or 0.04, etc. which defines the speed at which the blood sugar initially falls during a time interval. For older patients a lower sensitivity factor will cause the dosing rate to be adjusted in a slower manner whereas in younger patients there would be a more aggressive approach and a higher initial sensitivity factor would be selected. The sensitivity factor which is entered into processor 402 may be provided by the physician or caregiver.

Once the sensitivity factor is entered in block 68, which is the patient's sensitivity to insulin, the logic flow passes to target range 70 wherein the preferred blood-glucose target range for a particular patient is entered. The target range is the range which would be acceptable for the patient with respect to the blood glucose level. In general, there are options available to choose for an appropriate target range. The choice of an option is dependent upon a patient's physical state and may generally be between 70-100 mg/dL for a hyperosmoler patient. For a non-hyperosmoler patient the range may be between 120-160 mg/dL. For a pregnant female the range most often chosen is between 90-120 mg/dL. Where the patient's physical state is improving, the target range is between 100-140 mg/dL. The preferred target range is individualistic between patients and is chosen by the attending physician as a function of the aforementioned variables as well as a particular patient's history.

Once the blood-glucose target range has been inserted, the logic flows to logic block 40 on line 510 (shown in FIGS. 2B and 3). In block 40 the patient's initial blood glucose level is entered into processor 402 and the logic then flows to logic block 42 (in FIG. 2B). In block 42 the blood glucose level entered in block 40 is confirmed. If there is a confirmation then the logic flows to block 44 where various parameters external to the patient's parameters are entered such as the attending physician's (or nurse/caregiver) name and identification. The logic then flows to block 54 where identification of any changes in the patient's condition are shown which could affect the blood sugar level. Such changes are then listed and provided on display 406. Such changes may be the addition or discontinuation of specific medication, physical condition change such as a stroke or myocardial infarction or bleeding or any other condition which must be taken into account by the physician. Also, medications and nutritional changes which may require more or less insulin to offset any nutritional changes is taken into account in block 54.

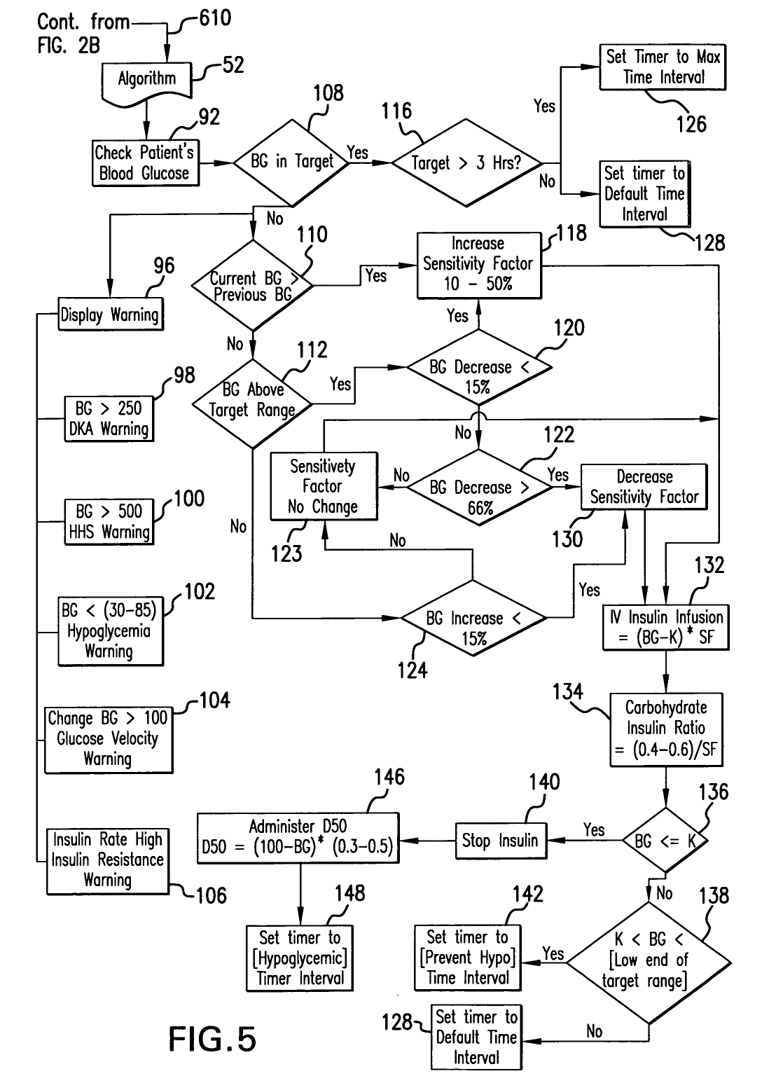

Once all the inputs are inserted into the computer, the logic flows to algorithm block 52 through information line 610 to determine the blood-glucose dosing rate. The algorithm block 52 and subcomponents of the logic flow are seen in FIG. 5. A display warning in block 96 is provided if the blood glucose is greater than 250 (which shows diabetic ketoacidosis) in block 98 or blood glucose is greater than 500 (warning for hyperglycemic/hyperosmolarity) in block 100; or blood glucose is lower than the range of 30-85 in block 102 (which provides for a hypoglycemia warning). Additionally, a warning is given for a change in the blood glucose level from the last time interval initiation if it shows a change of blood glucose greater than 100 in block 104 providing for a glucose velocity warning; and, if there is an insulin rate high insulin resistance warning in block 106. All of these are provided in the display warning 96 block and are provided directly to the user as a visual warning or may be transmitted to a server 412 or remote station 414 or some paging system or a text message or e-mail to be sent over the internet.

If no warning is necessary in blocks 96, 98, 100, 102, 104, or 106 the logic flows to decision block 108 from block 92 which determines whether the blood glucose level is in the target range which has previously been input in decision block 40 in FIG. 2B.

If the blood glucose is determined to be within the target range in decision block 108, logic flows to decision block 116 where it is determined whether the blood sugar level has been within the target range for more than a predetermined time (standardly being three hours). If the blood glucose level has not been within the target range within the predetermined time interval the timer is then set in block 128 to a default time interval (which may be one hour or two hours, etc.). In the event that the blood glucose level has been within the target range for the previous time interval then the logic flows to block 126 where the timer is set to some maximum time interval empirically dependent upon the physician or attending caregiver.

Referring back to decision block 108, if the blood glucose level is not within the target range then the logic flows to decision block 110 to determine whether the blood glucose level at this time is greater than the previous blood glucose level.

If the current blood glucose level is greater than the previous blood glucose level then the logic flows to block 118 where the sensitivity factor is increased between 10%-50% (the actual percentage being protocol dependent).

Having increased the sensitivity factor by a predetermined amount (either based upon the physician's or caregiver's empirical input or protocol), the flow then goes to block 132 where the dosing rate or IV insulin infusion rate is calculated. The calculation is made in accordance with the formula:
DR=(BG−K)×SF (1)
where:

DR=Dosing/Infusion Rate (units of insulin/time)

BG=patient blood glucose level (mg./dl.)

K=constant where 40<K<80

SF=sensitivity factor (protocol dependent)

and:

65<K<80 for capillary measurement

40<K<70 for arterial measurement

40<K<70 for venous measurement

65<K<80 for interstitial measurement

In the above formula, the constant K may also vary based on rate of blood glucose change and the target blood sugar range, however, such still being in accordance with the constant K being within the range 40<K<80.

In logic block 134 the carbohydrate insulin ratio is calculated in general as being 0.4-0.6 divided by a sensitivity factor. The carbohydrate insulin ratio is a number used to calculate how much insulin is needed to offset carbohydrate intake so that a patient's blood glucose level is not affected.

Once the carbohydrate insulin ratio is determined in block 134, the logic flows to decision logic block 136 where a determination is made as to whether the blood glucose is less than or equal to the constant K. If the blood glucose in block 136 is less than K then the logic flows to logic block 140 where insulin dosing is terminated. Instructions are then provided in block 146 to administer D50 which is dextrose 50% in accordance with the formula amount of D50=(100−BG)×(0.3-0.5). The timer is then set to the hypoglycemic time interval in block 148 where the hypoglycemic time interval is reset to a predetermined time interval which may be in general 30 minutes.

In the event that the blood glucose level is greater than K then the logic flows to decision block 138 where it is determined whether the blood glucose is within the range of K<BG<(low end of the preferred blood-glucose target range). If the BG is within the range then the logic flows to block 142 where the timer is set to a time interval to prevent hypoglycemia. If the BG is not within the range of K<BG<(low end of target range) then the logic flows to logic block 128 setting the timer to a default time interval.

In the event that the blood glucose is not within the target range in decision block 108 the logic flows to decision block 110 where a decision is made as to whether the current blood glucose level is greater than the previous blood glucose level at the previous time interval. If the current blood glucose level is determined to be less than the previous blood glucose level in decision block 110 the logic then flows to logic block 112 where a decision block is provided to determine whether the blood glucose level is above the target range. If the blood glucose level in block 112 is determined to be above the target range, the logic then flows to decision logic block 120 to determine whether the blood glucose decrease is less than 15% from the previous interval.

If the blood glucose decrease is less than 15% of the previous time interval, the logic flows to logic block 118 which provides for an increase in the sensitivity factor within the range of 10-50% which is empirically entered by the attending physician or caregiver. Once the sensitivity factor has been increased the logic then flows to block 132 as previously discussed to provide for the IV insulin infusion in accordance with the previous formula.

If the blood glucose decreases between the previous interval and the present time is equal to or greater than 15% than the logic flows to decision block 122 where the blood glucose decrease is determined as to whether it is greater than 66%. If the blood glucose level has decreased greater than 66% then the logic flows to block 130 where the sensitivity factor is decreased in accordance with the protocol of the physician or caregiver. Once the sensitivity factor decrease has been entered the logic then flows to block 132 to determine the insulin dosing rate in accordance with the previously derived formulas.

If the blood glucose decrease is equal to or less than 66% then the logic flows to block 123 and no sensitivity factor change is provided. Once no sensitivity factor change is inserted, the logic flow then once again passes to IV insulin infusion block 132.

Referring back to decision block 112, if the blood glucose level is below the target range, the data flow passes to decision block 124 where a decision is made as to whether the blood glucose increase between the previous interval and the present time is less than 15%. If the blood glucose increase between the last time interval and the current time is less than 15% then logic flows to block 130 where the sensitivity factor is decreased and the information flow then passes to block 132 for the IV insulin infusion calculation.

If the blood glucose increase is not less than 15% then the logic flow passes to block 123 where there is no sensitivity factor change and flow of data passes to block 132 for determining the dosage rate in accordance with the previous detailed formulas.

Returning to FIG. 2B and algorithm block 52, once the insulin dosing rate has been determined, a confirmation is displayed on display 406 as to the insulin and glucose infusion. At this point the attending physician or caregiver may override the dosing in decision block 46. If the algorithm dosing rate is confirmed and no override is provided in decision block 46 the logic passes to IV treatment information page block 18 which will be detailed in following paragraphs.

If it is determined to override the dosing rate as provided by algorithm block 52, the attending physician or caregiver enters the glucose/saline rate in block 48 and the logic passes to a decision block 56 where a display is provided to confirm the override. If the override is canceled, then the logic flows back to block 50 where a confirmation of the insulin and glucose infusion rate from algorithm 42 is confirmed. If override is confirmed in decision block 56, the logic flows back to block 50 for a confirmation of the insulin and glucose infusion as previously discussed.

Referring back to FIG. 2A, if in decision block 24 a standard intravenous route is to be performed, the logic flows to standard intravenous treatment block 28 where the patient information such as patient ID, weight, age, medications, disease state and further identification factors are inserted into microprocessor 402 through the display 406 and keyboard 416 or other touch screen input. Once the patient information has been entered in block 30 the user has the option to go back to standard intravenous treatment block 28 in order to modify the treatment.

However, once the information has been entered in block 30, the logic flows to flow block 32 where the insulin concentration and sensitivity factor for the patient is entered. Flow then continues to decision block 34 where it is determined whether a custom sensitivity factor is to be inserted by the user. A custom sensitivity factor may be entered in block 36 based upon the empirical knowledge of the physician or caregiver. In the event there is no custom sensitivity factor to be entered, the flow goes directly to selection of the target range infusion variables in flow block 38. Thus, whether a custom sensitivity factor is to be entered or a standard sensitivity factor is to be entered, the flow logic ends at information block 38 where a selection of the blood glucose target range is made.

From block 38 posing on information line 520 (to FIG. 2B), the blood glucose level of the patient is provided in flow block 40 in the same manner as derived from the express intravenous treatment block 26 previously discussed.

Once the blood glucose level of the patient has been entered, the logic flow passes to a confirmation of the blood glucose in block 42, entering caregiver's identification in block 44 and then to changes in condition in block 54 prior to passing to algorithm block 52.

The algorithm block 52 calculates the dosing rate of the insulin as previously discussed for the express intravenous treatment flow in block 26. Finally, in accordance with the previously discussed data flow, the information passes to confirmation of the insulin and glucose infusion in block 50 and then to the override dosing decision block 46 and decision block 56. Where there is no override dosing in decision block 46, logic flow passes to the intravenous treatment information page block 18.

Thus, whether in decision block 16 entitled “resume IV patient” where there is a resumption of the IV patient or in either the express intravenous treatment or standard intravenous treatment calculations, all information flows to IV treatment information page 18 as shown in FIGS. 2A and 2B. Further discussion of IV treatment information page 18 will be provided in following paragraphs.

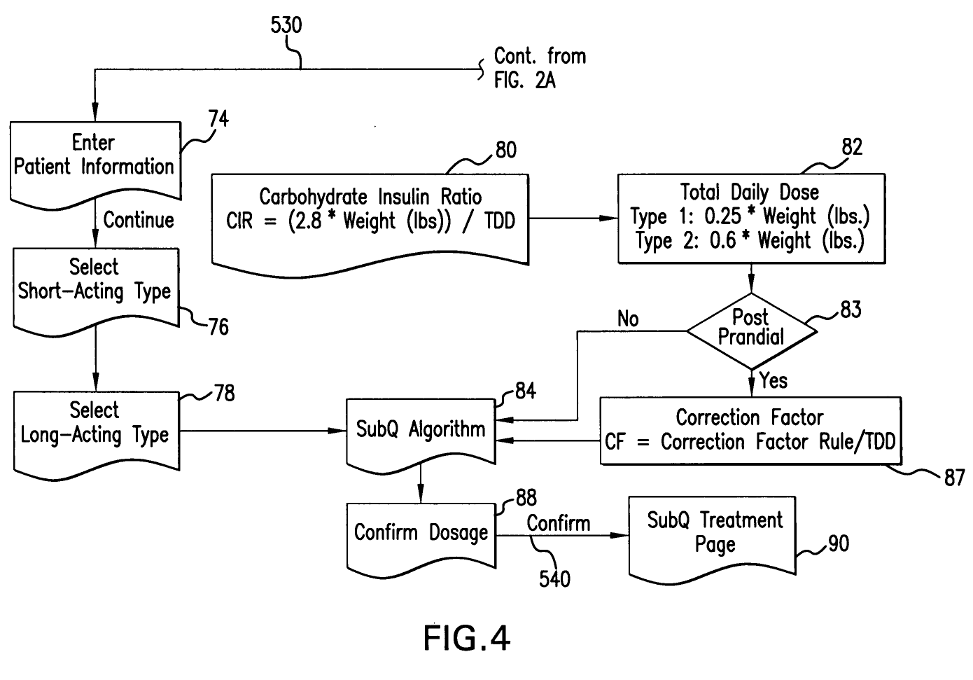

Referring back to FIG. 2A, what has previously been discussed is the follow on logic flow from decision block entitled “intravenous treatment” where a standard or express intravenous treatment has been entered. In the event that the treatment is to be subcutaneous, the information flow passes from intravenous treatment decision block 22 to subcutaneous block 20 and then enters FIG. 4 on information line 530.

Referring to FIG. 4, subcutaneous block 20 (shown on FIG. 2A) is provided for information to be entered as to the patient's physical parameters in block 74. Once the patient information has been entered in block 74, a selection of a short acting type subcutaneous treatment is provided in block 76 and a long acting type subcutaneous treatment is entered in block 78. At the physician's or caregiver's directions, both a short-acting type subcutaneous treatment and a long-acting type subcutaneous treatment may be entered.

From block 78, the information passes to the subcutaneous algorithm block 84. With regard to the subcutaneous algorithm block 84, the calculations made therein are provided in information block 80, information block 82 and information block 87 dependent upon a decision as to be made in decision block 83 as will be described in following paragraphs.

The subcutaneous algorithm block 84 acts on both a pre-prandial dosing rate calculation or on a post-prandial dosing rate.

Where there is a pre-prandial dosing rate to be provided to the patient the calculation is made in accordance with the following formulation:

$$DR = \frac{{C}_{H}}{CIR} \qquad ( 2 )$$

where: DR=Dosing Rate (units of insulin/time)

C_{H}=number of carbohydrates to be eaten (grams)

CIR=carbohydrate insulin ratio
where:

$$CIR = \frac{2.8 {xW}_{t}}{TDD} \qquad ( 3 )$$

where: Wₜ=patient weight (grams)

TDD=Total Daily Dosage of insulin (grams/day)

where: TDD=0.25×Wₜ for type 1 patient

TDD=0.6×Wₜ for type 2 patient

With regard to a post-prandial dosing rate the subcutaneous algorithm 84 calculates the dosing rate in accordance with the following formula:

$$DR = \frac{( BG - {T}_{BG} )}{CF} \qquad ( 4 )$$

where: BG=Blood Glucose Level of Patient (mg/dL)

T_{BG}=Target Blood Glucose Level of Patient (mg/dL)

CF=Correction Factor

$$CF = \frac{CFR}{TDD} \qquad ( 5 )$$

where: CFR=correction factor rule empirically chosen between 1500 and 2000

Thus, as far as the logic flow is concerned, the carbohydrate insulin ratio is calculated in block 80 and then the total daily dose (TDD) is calculated or provided in information block 82 dependent upon whether the patient is a Type 1 patient or a Type 2 patient. The Type 1 patient is for a patient who is insulin dependent and a Type 2 patient is for a patient which is non-insulin dependent, such terms well known in the art and discussed above. The information then passes to decision block 83 where it is determined whether this is a post-prandial treatment or a pre-prandial treatment. If it is not a post-prandial treatment the information passes directly back to subcutaneous algorithm block 84 and in the event it is a pre-prandial treatment decision in block 83 it passes to block 87 where a correction factor is provided to the algorithm and then the dosing rate is calculated based upon the correction factor, the blood glucose level, the target blood glucose level of the patient and the correction factor itself.

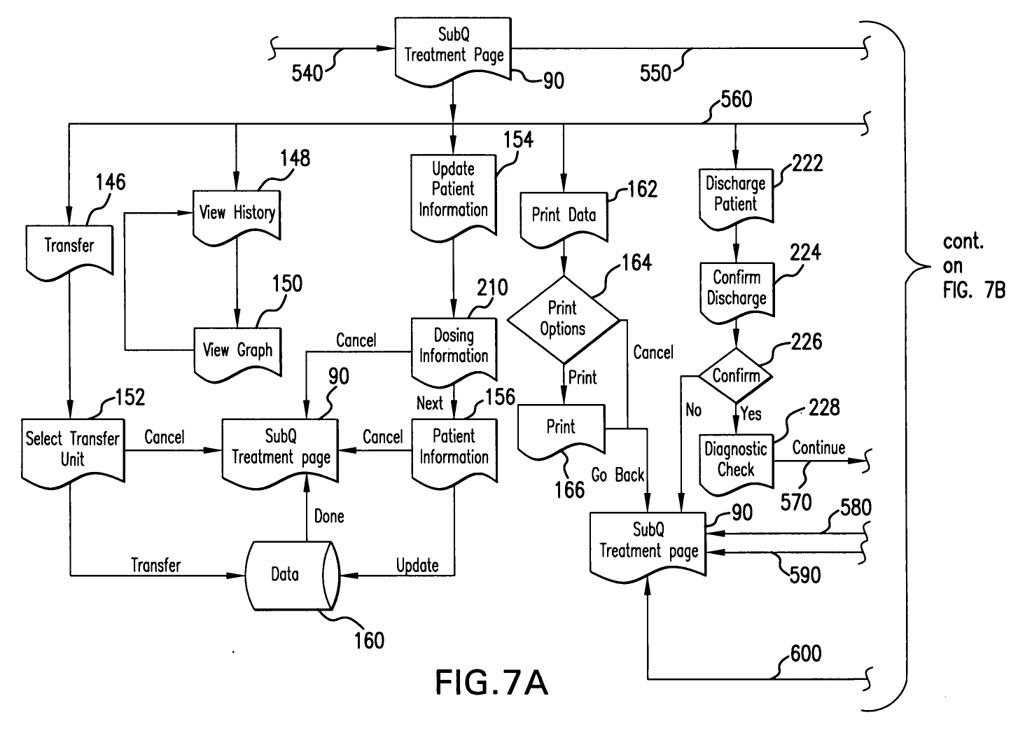

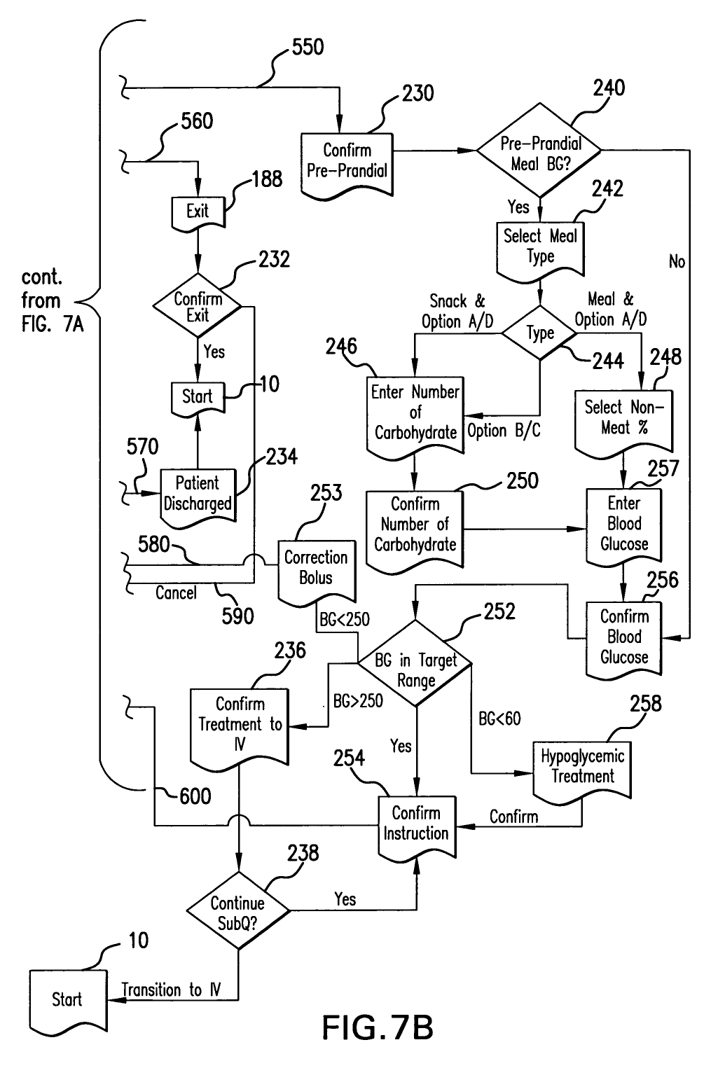

From the subcutaneous algorithm block 84, confirmation is made as to the dosage rate in block 88 which is displayed on display 406 for the physician or caregiver's review. Once this is confirmed, the information passes to the subcutaneous treatment page 90 on information line 540 (FIG. 7A), to be discussed in following paragraphs.

Referring back to FIGS. 2A and 2B, whether the resumption of the intravenous patient in decision block 16 is answered “yes” and the override dosing decision in block 46 is answered “no”, then information passes to the IV treatment information page 18. The IV treatment information page 18 provides for numerous options to be taken by the physician or caregiver as well as a decision as to whether a conversion to subcutaneous treatment is to be made from the intravenous treatment.

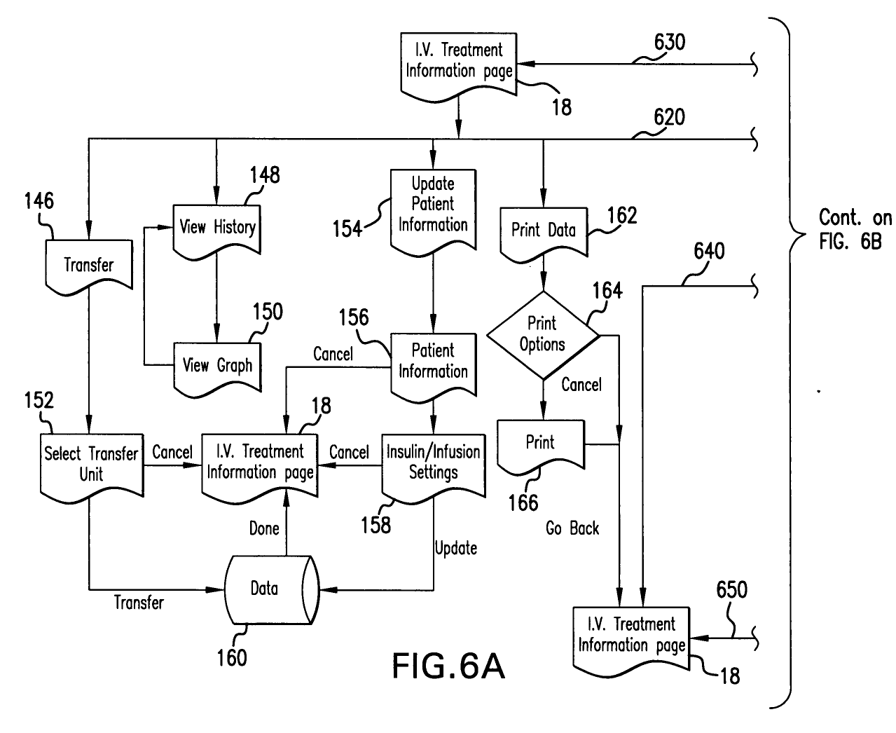

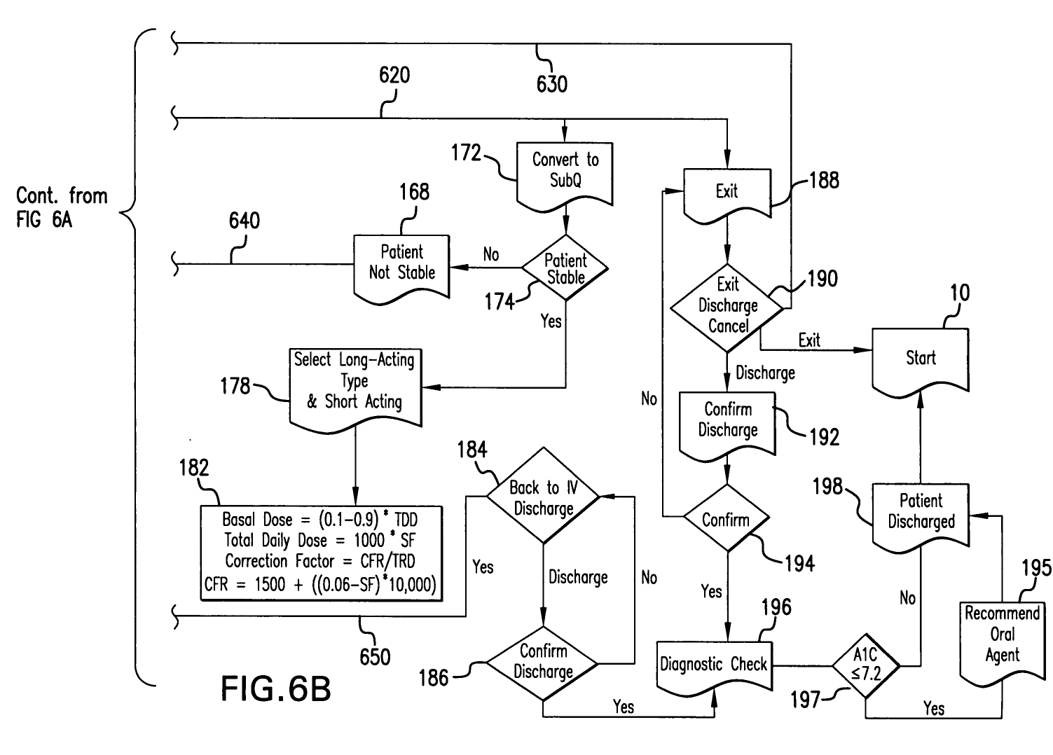

Referring now to FIGS. 6A and 6B, the intravenous treatment information page 18 is provided as an initialization point and the caregiver can choose to transfer a patient (information) in block 146 and selects a transfer unit in block 152 for transference of the data to the data block 160. The transfer information block 146 is provided to allow transfer of the information provided to the display 406, the remote station 414, to a server 412 or some other transfer unit. The data 160 is stored within the microprocessor 402 or some external transfer device.

The physician or caregiver may also view the history of the blood glucose level for the particular patient and other parameters of the patient in the view history block 148 and this can be transferred to a display graph in block 150.

Additionally, the patient information may be updated in block 154 and inserted into microprocessor 402 in patient information block 156 wherein patient information may be directly sent to the IV treatment information page 18 for viewing or insulin/infusion settings may be provided in block 158 and passed to data block 160.

Additionally, data from the IV treatment information page 18 may be passed to a print data information block 162 where a decision is made as to print options in decision block 164 where the information may be printed in block 166 or whether no print information is to be sent back to the information treatment information page block 18.

In some cases, the attending physician or caregiver may decide to convert the intravenous settings to a subcutaneous dosing rate through information line 620 to block 172 where the settings in microprocessor 402 are set to a subcutaneous dosing rate. The information then flows to a decision block 174 where it is determined whether the patient is stable or not stable. If the patient is not stable, information passes to information block 168 and then back to the intravenous treatment information page 18 on line 640.

If the patient is stable in decision block 174 information then passes to information block 178 where a selection is made as to a long-acting type dosing rate and/or a short-acting dosing rate.

Once the selection is made in block 178, information is then passed to the information block 182 where a calculation is made in accordance with the formula:

$$Basal dose = ( .1 - .9 ) \times TDD \\ Total Daily Dose ( TDD ) = 1000 \times SF \\ Correction Factor ( CF ) = \frac{CFR}{TDD} \\ CFR = 1500 + ( ( 0.06 - SF ) \times 10 \text{,} 000 ) \qquad ( 6 )$$

Information then passes to decision block 184 entitled “back to IV/discharge”. If it is decided to maintain the patient on an intravenous dosing rate the information then simply flows back to intravenous treatment information page 18 through information line 650.

If the decision is made to discharge the patient from subcutaneous dosing treatment in block 186 information passes to “confirm discharge” block 186.

Information is inserted into information block 196 “diagnostic check”. The information then flows to decision block 197 which determines whether the hemoglobin AIC is less than or equal to 7.2. If the hemoglobin AIC in decision block 197 is less then or equal to 7.2 the information passes to block 195 where an alert is displayed to the user recommending that the physician consider oral agent prescription for the patient. Information then passes to block 198 where the patient is discharged and returns to the start block 10. If the AIC is greater than 7.2 oral agent block 195 is bypassed and information simply enters patient discharged block 198.

Another branch of the intravenous treatment information page 18 in FIG. 6B is provided where the exit block 188 may be entered on line 620 which is a discharge patient block information data input. The decision to discharge goes to decision block 190 which gives the option to either “exit”, “discharge”, or “cancel”. If it is desired cancel this portion of the program the information passes back to intravenous treatment information page 18 on line 630 for new input to be inserted.

If the patient is to simply exit the program, the information passes back to the start block 10 for new insert of data as provided in FIG. 2A. If a discharge of the patient is to be made, the confirmation of discharge is made in block 192 and then passes to decision block 194 where a confirmation is required. If there is no confirmation of the discharge of the patient then the information passes back to exit block 188.

If confirmation of the discharge is made in block 194 the information passes once again to diagnostic check 196 and then through decision block 197, oral agent 195 and then to patient discharge block 198.

Referring now to FIGS. 7A and 7B there is provided a subcutaneous treatment page 90 which refers back to the subcutaneous treatment page on FIG. 4. In the subcutaneous treatment page 90 the user may once again transfer data in block 146 to a selected transfer unit in block 152 for transfer into some external data system 160. Similarly, the patient's history may be viewed in block 148 and a graph representation provided in block 150. The user's update information may be updated in block 154 with dosing information provided in block 210 and further patient information given in block 156 for passage to the subcutaneous treatment page 90 as shown in FIG. 7A.

The usual data options may be chosen by the user in the print data input block 162 and decision block 164 for either printing in block 166 on some external peripheral device or if there is no print the information simply passes back to the subcutaneous treatment page 90.

If a decision is made to discharge the patient in block 222 a confirmation of the discharge is made in block 224 and decision block 226 where if there is no confirmation of discharge of the patient, the information passes to the subcutaneous treatment page 90 and alternatively passes to the diagnostic check block 228 in the event of confirmation of the discharge. The patient information as to discharge is then passed to patient discharge block 234 through information line 570 and then back to the initial block “start” 10.

If in the subcutaneous treatment page 90 it is decided to exit the subcutaneous phase, the information passes to information block 188 through information line 560 and then to decision block 232 to confirm the exiting. If exit is confirmed the information passes to start block 10 and if is not confirmed then information passes back to subcutaneous treatment page 90 through information line 590 for display of information to the user.

In the event that dosing rate is to be determined with respect to pre-prandial or post-prandial dosing rates, information passes from the initial block 90 to conformation block 230 through information line 550 and decision block 240. In information block 230 the user enters whether the blood sugar is pre-prandial or post-prandial blood glucose levels. If the blood glucose level which has been entered is pre-prandial then the logic flows to block 242 where a selection of the meal type is made by the attending physician. The flow of data is determined in block 244 depending upon the type of meal as to whether it is a “snack” or “breakfast”, “lunch”, or “dinner” is made. If this is a snack type meal, the information moves to block 246 where the number of carbohydrates in the meal are entered and confirmed in information block 250.

Once the number of carbohydrates has been entered in block 250, the information is directed to block 257 where the blood glucose level is entered. The confirmation of the blood glucose is made in block 256 and then the information moves to decision block 252 to determine whether the blood glucose is within the target range. If the blood glucose is within the target range then the information moves to block 254 where the instructions are confirmed and all information is then passed back to subcutaneous treatment page 90 through line 600. If the blood glucose is not within the target range but the blood glucose is less than 60 then the information moves to hypoglycemic block 258. Once the hypoglycemic treatment is confirmed in block 254 the information then passes back to subcutaneous treatment page 90 through information line 600.

If the blood glucose is greater than 250 as found in decision block 252, information is directed to the “confirm treatment to IV” block 236 and then passes to decision block 238 where it is decided to either continue the subcutaneous treatment or to transition back to IV. If it transitions back to IV the information simply passes back to start block 10.

If the decision is made to continue the subcutaneous treatment, the information then passes to confirm instruction block 254 and then back to the subcutaneous treatment page 90.

Returning back to decision block 252, if the blood glucose is not in the target range but is less than 250, the information is passed to correction bolus block 253 where a correction bolus is provided by the attending physician in an empirical manner. Once this is completed, the information then passes to subcutaneous treatment page 90 through information line 580.

Going back to decision block 244 where the type of meal is decided by attending physician or caregiver, if the meal is to be a breakfast, lunch or dinner, the information block 248 selects the percentage of the meal which is non-meat. The blood glucose level of the patient is entered in block 251 as previously discussed and the conformation of the blood glucose is made in block 256. Once again the information passes to decision block 252 where it is determined what range the blood glucose is with respect to the target range. If the blood glucose is less than 250 but external to the target to the target range the information then passes to correction bolus information block 253 and then back to the subcutaneous treatment page 90 on information line 580.

If in block 252 the blood glucose is greater than 250 information passes once again to the “conformation treatment to IV block” 236 and then to the decision block 238 where it is either decided to continue the subcutaneous treatment or whether there is a transition to intravenous to be made as previously described.

If the blood glucose level is less than 60 the information then passes to hypoglycemic treatment block 258 with a confirmation of the instruction being made in block 254 and then passage to the subcutaneous treatment page 90 as previously discussed.

It would be appreciated by those skilled in the art that changes could be made to the embodiment described above without departing from the broad inventive concept thereof. It is understood, therefore, that this invention is not limited to the particular embodiments disclosed, but to cover modifications within the spirit and scope of the present invention as defined by the appended claims.

What is claimed is:

1. A system for measuring and predicting an insulin dosing rate to bring a patient's blood glucose level into a preferred target range comprising:
(a) a processor for actuating a predetermined blood glucose computer program for measurement and prediction of said patient's blood glucose level;
(b) input mechanism for inputting a preferred target range of said patient's blood glucose level and patient input data including patient weight into said processor;
(c) means for calculating the optimum insulin dosing rate for said patient based upon a type of insulin dosing chosen from the group of intravenous dosing and subcutaneous dosing of said patient;
(d) a display mechanism for displaying patient dosing parameters;
(e) an alarm mechanism for alerting a user when said patient's blood glucose level is external the preferred patient blood glucose target range.

2. A system for measuring and predicting an insulin dosing rate as recited in claim 1 where said means for calculating the optimum insulin dosing rate includes means for designating whether the insulin dosing rate calculations are based upon subcutaneous dosing or intravenous dosing.

3. A system for measuring and predicting an insulin dosing rate as recited in claim 2 where said means for calculating the optimum insulin dosing rate includes a means for designating said intravenous dosing is based upon a standard intravenous dosing or an express intravenous dosing.

4. A system for measuring and predicting an insulin dosing rate as recited in claim 2 whereby said means for calculating the optimum insulin dosing rate is in accordance with the formula:
DR=(BG−K)×SF
where: BG=patient blood glucose level
K=constant where 40<K<80
SF=sensitivity factor (protocol dependant)
and:
65<K<80 for capillary measurement
40<K<70 for arterial measurement
40<K<70 for venous measurement
65<K<80 for interstitial measurement.

5. A system for measuring and predicting an insulin dosing rate as recited in claim 1 where said means for calculating the optimum insulin dosing rate based upon subcutaneous dosing includes:
(a) the means for calculating the subcutaneous dosing based upon pre-prandial parameters of the patient; and,
(b) the means for calculating the subcutaneous dosing rate based upon post-prandial conditions of said patient.

6. A system for measuring and predicting an insulin dosing rate as recited in claim 1 including means for switching said patient's dosing rate from an intravenous dosing rate to a subcutaneous dosing rate.

7. A system for measuring and predicting an insulin dosing rate as recited in claim 1 wherein said alarm mechanism for alerting a user includes an audio signal emitted by the processor when said patient's blood glucose level is external to a standard target range.

8. A system for measuring and predicting an insulin dosing rate as recited in claim 1 where said alarm mechanism includes a visual mechanism provided on the display mechanism for displaying a signal responsive to said patient's blood glucose level being external to the preferred patient blood glucose target range.

9. A system for measuring and predicting an insulin dosing rate as recited in claim 1 where said input mechanism for inputting a preferred target range of said patient's blood glucose level includes a keyboard electrically coupled to a display for entry of said patient's biological parameters.

10. A system for measuring and predicting an insulin dosing rate as recited in claim I where said display mechanism includes a touch screen processor display system for inputting said patient's biological parameters.

11. A method of optimally measuring and predicting an intravenous insulin dosing rate to bring the patient's blood glucose level into a target range including the steps of:
(a) establishing a processor for actuating an intravenous computer program for measurement and prediction of said patient's glucose level;
(b) inputting predetermined patient data into said processor including a preferred blood glucose target range for said patient;
(c) calculating the optimum dosing rate of said insulin in accordance with:
DR=(BG−K)×SF
where: BG=patient blood glucose level
K=constant where 40<K<80
SF=sensitivity factor (protocol dependant)
and:
65<K<80 for capillary measurement
40<K<70 for arterial measurement
40<K<70 for venous measurement
65<K<80 for interstitial measurement.

12. The method as recited in claim 11 includes the step of measuring said patient's blood glucose level after a predetermined time interval.

13. The step method as recited in claim 12 which is followed by the step of adjusting the patient's insulin dosing rate after said predetermined time interval.

14. The method as recited in claim 13 which is followed by the step of repeating step (c) of claim 11.

15. The method as recited in claim 14 where the step of adjusting includes the step of increasing the K between 5-15 when said patient's blood glucose level is greater than an upper level of said target range and inserting said K value into said processor.

16. The method as recited in claim 14 where the step of adjusting includes the step of decreasing the K between 5-15 when the patient's blood glucose level is less than a lower level of said target range and inserting said K value into said processor.

17. A method of measuring and predicting a subcutaneous insulin dosing rate to bring a patient's blood glucose level into a preferred target range including the steps of:
(a) establishing a processor for actuating a subcutaneous computer program for measurement and prediction of said patient's blood glucose level;
(b) establishing said preferred target range of said patient's blood glucose level;
(c) inputting predetermined patient data into said processor including said patient target range, said patient's weight; and,
(d) calculating the optimum insulin dosing rate dependent on whether the calculation is being made pre-prandial or post-prandial.

18. The method as recited in claim 17 wherein the step of calculating includes the step of determining whether the dosing is being applied pre-prandial or post-prandial.

19. The method as recited in claim 18 where the step of determining is followed by the step of calculating a pre-prandial dosing rate in accordance with:
$$DR = \frac{{C}_{H}}{CIR}$$
where: C_{H}=number of carbohydrates to be eaten (grams)
CIR=carbohydrate insulin ratio
where:
$$CIR = \frac{2.8 {xW}_{t}}{TDD}$$
where: Wₜ=patient weight
TDD=Total Daily Dosage of insulin
Where: TDD=0.25×Wₜ for type 1 patient
TDD=0.6×Wₜ for type 2 patient.

20. The method as recited in claim 17 wherein the step of determining is followed by the step of calculating a post-prandial dosing rate in accordance with:
$$DR = \frac{( BG - {T}_{BG} )}{CF}$$
where: BG=Blood Glucose Level of Patient (mg/dl)
T_{BG}=Target Blood Glucose Level of Patient (mg/dl)
CF=Correction Factor
$$CF = \frac{CFR}{TDD}$$
where: CFR=correction factor rule empirically chosen between 1500 and 2000
